(19) United States
(12) Patent Application Publication
(10) Pub. No.: US 2003/0215412 A1
(43) Pub. Date: Nov. 20, 2003

(54) Induction of hair growth with vascular endothelial growth factor

(75) Inventors: Jacob Waugh, Palo Alto, CA (US); Michael Dake, Stanford, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Essentia Biosystems, Inc., Palo Alto, CA

(21) Appl. No.: 10/370,830

(22) Filed: Feb. 21, 2003

Related U.S. Application Data
(63) Continuation-in-part of application No. 09/910,432, filed on Jul. 20, 2001.
(60) Provisional application No. 60/359,400, filed on Feb. 21, 2002.
(60) Provisional application No. 60/220,244, filed on Jul. 21, 2000.

(51) Int. Cl. A61K 38/18; A61K 7/06; A61K 7/11

(52) U.S. Cl. 424/70.14; 514/12

(57) ABSTRACT

A method for inducing or stimulating new hair growth, increasing hair growth or preventing hair regression in a mammalian subject in need of or desirous of such treatment is provided that comprises administering to the subject a pharmaceutically or cosmeceutically effective amount of a composition comprising vascular endothelial growth factor (VEGF), a VEGF receptor agonist, a prodrug form of the foregoing or a salt form thereof in a non-covalent association complex with a positively-charged backbone having a plurality of attached efficiency groups.

### CROSS-REFERENCE TO RELATED APPLICATIONS

This is a continuation-in-part of application Ser. No. 09/910,432 filed Jul. 20, 2001, which claims the priority of U.S. provisional application No. 60/220,244 filed Jul. 21, 2000. In addition, this Application claims the priority of U.S. provisional application No. 60/359,400 filed Feb. 21, 2002.

### BACKGROUND OF THE INVENTION

Vascular endothelial growth factor (VEGF or V-EGF) is an endothelial cell specific mitogen that is essential for endothelial cell differentiation (vasculogenesis) and for the sprouting of new capillaries from preexisting vessels (angiogenesis) (Carmeliet, P., et al., Nature 380:435-439 (1996); Ferrara, N. Eur. J. Cancer. 32A:2413-2422 (1996)). VEGF (VEGF-A) is a distant relative of platelet-derived growth factor (PDGF) (Zachary, I. Am. J. Physiol. Cell Physiol. 280:C1375-C1386 (2001)). It is a member of a family of related growth factors that currently includes VEGF-B, -C, -D, and -E and placenta growth factor (PIGF) (Achen, M. G., et al., Proc. Natl. Acad. Sci. USA 95:548-553 (1998); Ferrara, N., Eur. J. Cancer 32A:2413-2422 (1996), Jeltsch, M., et al., Science 276:1423-1425 (1997); Olofsson, B., et al., Proc. Natl. Acad. Sci. USA 93:2576-2581 (1996); Petrova, T. V., et al., Exp. Cell. Res. 253:117-130 (1999)).

The human vascular endothelial growth factor gene contains eight exons. Alternative splicing of VEGF mRNA gives rise to at least five different isoforms of 121, 145, 165, 189, and 206 amino acid residues (Ferrara, N., Eur. J. Cancer 32A:2413-2422 (1996); Poltorak, Z., et al., J. Biol. Chem. 272:7151-7158 (1997); Risau, W. Nature 386:671-674 (1997)). Exons 1-5 encode the core regions essential for binding to the receptors VEGF receptor 1 (VEGFR1) and VEGF receptor 2 (VEGFR2). Exon 6 encodes a region rich in basic amino acid residues and is a presumed heparin-binding domain. Exon 7 is implicated in binding to both heparin and neuropili-1 (NP-1). The vital role of VEGF in embryonic blood vessel development has been confirmed by the finding that targeted inactivation of only a single allele of VEGF in mice causes a lethal impairment of endothelial cell differentiation, development of the primitive vascular plexus and angiogenesis (Carmeliet, P., et al., Nature 380:435-439 (1996)).

Two distinct receptors with intrinsic tyrosine kinase activity (RTKs) have been identified for VEGF, VEGFR1 (Flt-1) and VEGFR2 (KDR/Flk-1). The two VEGF RTKs share approximately 44% amino acid homology with each other (Neufeld, G., et al., FASEB J. 13:19-22 (1999); Petrova, T. V., et al., Exp. Cell Res. 253:117-130 (1999)). A third receptor, VEGFR3 (Flt-4), binds VEGF-C and -D and does not bind VEGF-A (Booth, R. F., et al. Atherosclerosis 76:257-268 (1989); Joukov, et al., EMBO J. 15:290-298)). The three VEGF receptors are structurally related to the PDGF family of receptor tyrosine kinases (class III). They all have a similar domain structure characterized by cytoplasmic regions with an insert sequence within the catalytic domain, a single hydrophobic transmembrane domain and seven immunoglobulin-like domains in the extracellular regions (Zachary, I. Am. J. Physiol. Cell Physiol. 280:C1375-C1386 (2001)). In addition, neuropilin-1 (NP-1) was recently identified as another for VEGF (Soker, S., et al., Cell 92:735-745 (1998)). Neuropilin-1 is a non-tyrosine kinase transmembrane receptor with a short cytoplasmic tail and a large extracellular domain (Petrova, T. V., et al., Exp. Cell. Res. 253:117-130 (1999)). Earlier research identified that NP-1 is a receptor for the semaphorin/collapsing family of polypeptides implicated in axonal guidance (Kitsukawa, T., et al., Development 121:4309-4318 (1995)). Studies show that overexpression of NP-1 in mice results in diverse vascular abnormalities, including excess capillaries and blood vessels, and in malformation of the heart (Kitsukawa, T., et al., Neuron 19:995-1005 (1997)). NP-1 knockout mice display impaired neural vascularization, defects in the aorta and other large blood vessels, and aberrant yolk sac vascularization (Kawasaki, T., et al., Development 126:4895-4902 (1999)). There is no known signaling function for NP-1 and due to its short cytoplasmic tail and ability to bind a variety of semaphorins with equal affinity but with each having different biological activities, NP-1 is thought to be a docking receptor for VEGFR2 and not a functional receptor (Zachary, I. Am. J. Physiol. Cell Physiol. 280:C1375-C1386 (2001)).

VEGF has been found to play a role in vascular protection in the adult vasculature. Perivascular VEGF gene transfer inhibits neointima formation in a non-endothelial injury rabbit carotid artery model (Booth, R. F., et al., Atherosclerosis 76:257-268 (1989); Laitinen, M., et al., Hum. Gene Ther. 8:1645-1650 (1997); Laitinen, M., et al., Hum. Gene Ther. 8:1737-1744 (1993); Soma, M. R., et al., Arterioscier. Thromb. 13:571-578 (1993)). VEGF gene transfer with the use of the collar as a gene delivery reservoir was found to inhibit neointimal smooth muscle cell (SMC) hyperplasia in the absence of angiogenesis (Laitinen, M., et al., Hum. Gene Ther. 8:1737-1744 (1993)). Nitro-L-arginine methyl ester (L-NAME), an endothelial nitric oxide synthase (eNOS) inhibitor, prevented VEGF-mediated inhibition of neointima formation (Laitinen, M., et al., Hum. Gene Ther. 8:1737-1744 (1993)). This suggests the involvement of the nitric oxide pathway in VEGF neointima inhibition. Several studies have established that VEGF stimulates endothelial production of nitric oxide (NO) and prostacylin (PG12) (Horowitz, J. R., et al., Arterioscler. Thromb. Vasc. Biol. 17:2793-2799 (1997); Kroll, J., et al., Biochem. Biophys. Res. Commun. 265:636-639 (1999); Ku, D. D., et al., Am. J. Physiol. Heart Circ. Physiol. 265:H586-H592 (1993); Laitinen, M., et al., Hum. Gene Ther. 8:1737-1744 (1993); Murohara, T., et al., Circulation 97:99-107 (1998); Servos, S., et al., Cardiovasc. Res. 41:509-510 (19939); Tsurumi, Y., et al., Nat. Med. 3:879-886 (1997)). NO and PG1₂ have antiproliferative effects in SMCs and anti-platelet actions. In addition, NO inhibits leukocyte interaction with endothelium. Both NO and PG1₂ also mediate angiogenic and permeability-increasing effects of VEGF. VEGF-induced vascular permeability appears dependent on both NO production and prostaglandin production (Murohara, T., et al., Circulation 97:99-107 (1998)). In mice lacking the eNOS gene, impaired angiogenesis was not improved by administration of VEGF, which suggests that eNOS is downstream from VEGF (Murohara, T., et al., J. Clin. Invest. 111:2567-2578 (1998)). Promoting eNOS activity by administration of L-arginine accelerates in vivo angiogenesis (Murohara, T., et al., J. Clin. Invest. 111:2567-2578 (1998)). Adhesion molecule expression and leukocyte adhesion are important triggers during the early stages of atherosclerosis, which suggests that VEGF-induced NO synthesis may have an anti-inflammatory effect with the potential to protect against proatherogenic factors (Zachary, I. Am. J. Physiol. Cell Physiol. 280:C1375-C1386 (2001)).

In addition, VEGF also plays a role in the dermis. Researchers have demonstrated that vascular endothelial growth factor is of major importance for skin vascularization. Expression of VEGF is increased in hyperplastic epidermis of psoriasis (Petrova, T. V., et al., Exp. Cell Res. 253:117-130 (1999)), in wound healing (Brown, L. F., et al., J. Immunol. 154:2801-2807 (1995)), and in other skin diseases characterized by enhanced angiogenesis (Brown, L. F., et al. J. Exp. Med. 176:1375-1379 (1992); Brown, L. F., et al., J. Invest. Dermatol. 104:744-749 (1995)).

The hair follicle undergoes distinct cyclic expansion and regression that leads to rapidly changing needs for its vascular support. Adequate supply of blood is a prerequisite for normal cell growth and differentiation. It also seems to be of fundamental importance in the active processes of cell growth. Dermal papilla of the hair follicle as wells as the bulge present a well developed vascularization, therefore providing optimal growth conditions. The hair follicle undergoes a life-long cyclic transformation. There are three phases of the hair growth cycle: anagen, catagen and telogen. The anagen phase is involved with rapid proliferation of follicular keratinocytes and elongation and thickening of the hair (Yano, K., et al., The J. Clin. Invest. 107:409-417 (2001)). After anagen is completed, the hair enters the catagen phase. In the catagen phase, the matrix cells gradually stop dividing and eventually keratinize. This phase is short and usually lasts about 2-3 weeks. When full keratinization is achieved, the hair enters the last phase of the cycle, telogen. During the telogen (resting) phase, keratinized hair falls out, and a new matrix is gradually formed from the stem cells in the basal layer of the outer epithelial root sheath bulge (Jankovic, S. M., et al., Dermatology Online Journal 4(1):2). Afterwards, a new hair starts to grow and the follicle is back in the anagen phase.

Numerous studies have stressed that hair vasculature undergoes distinct cyclic expansion and regression that leads to rapidly changing needs for vascular support. Growing hair follicles are surrounded by blood vessels that have been postulated as arising from the deep dermal vascular plexus. Moreover, modulation of skin vascularization and perfusion has been previously observed during the hair cycle and in some human diseases characterized by hair loss (Yano, K., et al., The J. Clin. Invest. 107:409-417 (2001)). It has been observed that anagen human hair follicles are highly vascularized while most capillaries collapse and disappear during the catagen phase (Kozlowska, U., et al., Arch. Dermatol. Res. 290:661-668 (1998)). Studies have demonstrated that anagen hair follicles possess angiogenic properties in experimental in vivo models of angiogenesis (Kozlowska, U., et al., Arch. Dermatol. Res. 290:661-668 (1998)). Also, primary changes at the beginning of the catagen phase occur around the small vessels at the dermal papilla (Kozlowska, U., et al., Arch. Dermatol. Res. 290:661-668 (1998)). Although not fully confirmed, it has been thought that the reason hair follicles enter the catagen phase may be due to decreased vascularization (Parakkal, P. F., Hair and Hair Diseases, Springer Verlag, Berlin, 99-116 (1990)).

Pronounced angiogenesis occurs during murine hair follicle cycling (Yano, K., et al., The J. Clin. Invest. 107(4):409-417 (2001)). Experiments have demonstrated that overexpression of VEGF in follicular keratinocytes resulted in accelerated hair regrowth and in increased size of hair follicles (Yano, K., et al., The J. Clin. Invest. 107:409-417 (2001)). This result provides the first direct evidence that promotion of angiogenesis can promote hair growth and also hair thickness. Evidence also suggested that the effects of VEGF were mediated indirectly though induction of perifollicular angiogenesis. Systemic neutralization of VEGF significantly delayed hair regrowth and resulted in diminished perifollicular vascularization and reduced size of hair follicles (Yano, K., et al., The J. Clin. Invest. 107:409-417 (2001)). These findings show that normal murine follicle growth and cycling are dependent on angiogenesis induced by VEGF. In the past, impaired vascularization of the hair follicle has been suggested to play an important role in the pathogenesis of disorders characterized by hair loss (Goldman, C. K., et al., J. Invest. Dermatol. 104 (Suppl. 1): 18S-20S (1995)). These disorders include androgenetic alopecia (male-pattern hair loss) where baldness is associated with miniaturization of genetically predisposed hair follicles (Paus, R., et al., N. Engl. J. Med. 341:491-497 (1999); Cormia, F. E., et al, Arch. Dermatol. 84:772 (1961)).

A number of compositions and methods are known for promoting hair growth and/or treating alopecia. For example, U.S. Pat. No. 6,262,105 to Johnstone describes the use of prostaglandins. U.S. Pat. No. 6,288,112 to Seki, et al, describes the use of pyrethroids. U.S. Pat. No. 6,333,057 to Crandall describes topical compositions for increasing hair growth. However, the '057 compositions require an anti-androgen and a co-enzyme in addition to a penetrating agent. For many subjects, the use of anti-androgens is neither attractive nor desirous. In terms of delivery methods, U.S. Pat. No. 5,733,572 to Unger, et al., describe gas filled lipid-containing microspheres containing 50% gas in the interior of the spheres, effectively reducing the amount of available active component.

Despite the advances in understanding biological processes involved in hair growth and the role that VEGF may play in those processes, there remains a need for compositions that can be easily applied and provide stimulation for new hair growth or prevention of hair regression. Surprisingly, the present invention provides such compositions as well as methods for their use. Additional features and advantages will become apparent to those skilled in the art from the following description and claims.

### BRIEF SUMMARY OF THE INVENTION

In view of the above, the present invention provides, in one aspect, a method for inducing or stimulating new hair growth, increasing hair growth or preventing hair regression in a mammalian subject in need of or desirous of such treatment. More particularly the present invention contemplates a method comprising administering to the subject a pharmaceutically or cosmeccutically effective amount of a composition comprising vascular endothelial growth factor (VEGF), a VEGF receptor agonist, a prodrug form of the foregoing or a salt form thereof in a non-covalent association complex with a positively-charged backbone having a plurality of attached efficiency groups. Typically, the amount of vascular endothelial growth factor (VEGF) or VEGF receptor agonist in the non-covalent association complex will be that amount which is effective to induce or stimulate new hair growth, increase hair growth or prevent hair regression in a subject. As contemplated herein, the subject is preferably a mammalian subject. The method is suitable for use in a wide variety of instances, such as for restorative purposes, cosmeceutical or cosmetic purposes, clinical or prophylactic purposes, etc., as will be readily be apparent to one skilled in the relevant art.

In another aspect, the present invention provides medicaments comprising vascular endothelial growth factor (VEGF), a VEGF receptor agonist, a prodrug form of the foregoing or a salt form thereof in a non-covalent association complex with a positively-charged backbone having a plurality of attached efficiency groups for the induction of hair growth. More particularly, the present invention contemplates medicaments comprising a pharmaceutically or cosmeceutically acceptable carrier and an effective amount of vascular endothelial growth factor (VEGF), a VEGF receptor agonist, a prodrug form of the foregoing or a salt form thereof in non-covalent association with a complex of a positively-charged backbone having a plurality of attached efficiency groups.

In yet another aspect, the invention comprises the use of vascular endothelial growth factor (VEGF), a VEGF receptor agonist, a prodrug form of the foregoing or a salt form thereof in non-covalent association with a complex of a positively-charged backbone having a plurality of attached efficiency groups in the preparation of a medicament for the stimulation of new hair growth, to increase hair growth or for the prevention of hair regression in a mammalian subject.

In a further aspect, the invention provides a kit for inducing or stimulating new hair growth, increasing hair growth or preventing hair regression in a mammalian subject, the kit comprising a composition and a container, the composition comprising a pharmaceutically or cosmeceutically acceptable carrier and an effective amount of vascular endothelial growth factor (VEGF), a VEGF receptor agonist, a prodrug form of the foregoing or a salt form thereof in non-covalent association with a complex of a positively-charged backbone having a plurality of attached efficiency groups.

In the above aspects, in one preferred embodiment, the composition comprises a multi-component biological transport system as described below.

### BRIEF DESCRIPTION OF THE DRAWINGS

FIGS. 1-2 provide photographs at 100× magnification for skin samples obtained from two mice of Group A that were treated with Test Solution A as described in Example 1.

FIGS. 3-4 provide photographs at 100× magnification for skin samples obtained from two mice of Group B that were treated with Test Solution B as described in Example 1.

FIGS. 5-6 provide photographs at 100× magnification for skin samples obtained from two mice of Group C that were treated with Test Solution C as described in Example 1.

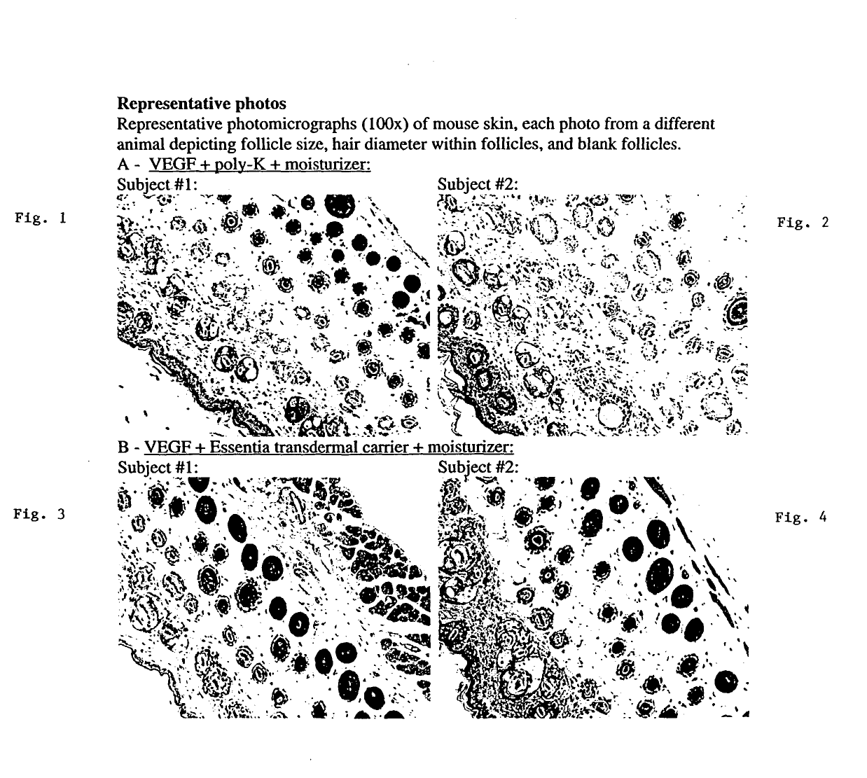

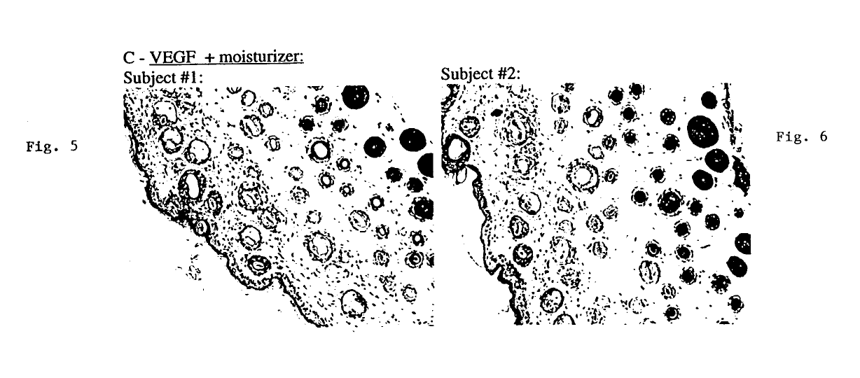

### DETAILED DESCRIPTION OF THE INVENTION

Medicaments that promote or enhance the rate of hair growth or stimulate an increase in follicle size and activity can be desirable in a number of situations. The compositions of the present invention can be appropriate for use in any situation where a subject is in need or desirous of hair growth. Among such situations include accidental injury, bum wound, damage to hair follicles, radiation or chemotherapy treatment site, surgical trauma, incisional wound, donor site wound from skin transplant, ulcer and alopecia, or in order to modify physical appearance. In humans especially, alopecia is often referred to as androgenic alopecia or androgen-dependent hair loss. Androgenic alopecia refers to an autosomal disorder that begins in puberty in genetically disposed individuals. Androgenic alopecia is also known as hereditary baldness, male pattern baldness and seborrheic alopecia. Androgenic alopecia can occur in males and females. When used alone, the term “alopecia” is understood herein to refer to hair loss in general, caused by any of a variety of circumstances, including the foregoing.

As discussed above, it has recently been found that vascular endothelial growth factor (VEGF or V-EGF), also known as vascular permeability factor (VPF), can be used to promote angiogenesis, increase the expression of endothelial nitric oxide synthase (eNOS) and stimulate the release of prostacyclin. Without being bound by theory, it has been proposed that the promotion or enhancement in the rate of hair growth or stimulation in an increase in follicle size and activity can be occasioned by the delivery of VEGF or VEGF receptor agonists to the high-affinity tyrosine kinase binding receptors that have been identified for VEGF, namely VEGFR1 (FLT-1) and VEGFR2 (KDR/FLK-1) or other receptors not currently recognized. Accordingly, it has now been found that VEGF, VEGF derivatives, VEGF fragments, VEGF receptor agonists, prodrug forms of the foregoing as well as salt forms thereof can be directly incorporated into compositions that will enable VEGF to penetrate the skin. Remarkably, VEGF can be delivered in an effective quantity to enhance the rate of hair growth and to stimulate an increase in follicle size and activity. In combination with an effective transdermal delivery platform, as described herein, it has now been shown that even smaller amounts of VEGF can be effective in providing benefits in inducing hair growth. With larger doses, VEGF can also provide hair inducing benefits with a reduced or even obviated need for a transdermal delivery platform.

Methods

The present invention provides, in one aspect, a method for inducing or stimulating new hair growth, increasing hair growth or preventing hair regression in a mammalian subject in need of or desirous of such treatment, comprising administering to the subject a pharmaceutically or cosmeceutically effective amount of a composition comprising vascular endothelial growth factor (VEGF), VEGF derivatives, VEGF fragments, VEGF receptor agonists, endogenous VEGF receptor agonist agents or prodrug forms of the foregoing or salt forms thereof. VEGF as well as VEGF derivatives and substituted forms of VEGF are suitable for use in the compositions of the present invention. For sake of convenience, the foregoing moieties, i.e., VEGF, VEGF derivatives, VEGF fragments, VEGF receptor agonists, prodrug forms of the foregoing and their respective salt forms are collectively referred to as “VEGF” or “VEGF compounds” in discussions below. The inventive VEGF compounds described herein are provided in a non-covalent association complex with a positively-charged backbone that has a plurality of attached efficiency groups, which may in turn be included in a multicomponent biological transport system. As is understood herein, the combination of a positively-charged backbone having a plurality of attached efficiency groups and a non-covalently associated VEGF compound is referred to as a “VEGF complex.”

The term “endogenous VEGF receptor agonist agent” as used herein is understood to relate to substances for increasing endogenous VEGF receptor agonist levels in situ. Examples of suitable endogenous VEGF receptor agonist agents for use with the present invention include estrogen and prostaglandin E2. In addition to the foregoing, VEGF receptor agonist prodrugs can also be used with the methods of the present invention. Prodrugs are well known in the art and include inactive drug precursors which, when activated by changes in temperature or pH, metabolism or metabolizing enzymes, pressure, presence or absence of oxygen etc., form active drugs. Thus VEGF receptor agonist prodrugs can also increase VEGF receptor agonist levels in situ, and are therefore regarded herein as one form of endogenous VEGF receptor agent. Accordingly, references to VEGF or VEGF receptor agonists herein are also to be regarded as including prodrug forms of VEGF or VEGF receptor agonists, respectively.

The terms “inducing”, “stimulating” or “increasing” as used in conjunction with “hair growth” herein is understood to relate to a retention in the number of existing hair follicles, an increase in hair follicle cross-sectional area, an increase in the number of hair follicles or the regrowth of hair follicles, resulting in an increase in the fullness, length, texture, or thickness of existing hair. A “receptor agonist” is used herein to refer to any ligand, especially a drug or hormone, that binds to a receptor and thereby alters the proportion of receptors that are in active form. The result is a biological response. A VEGF receptor agonist is an agonist of the VEGF high-affinity binding receptors, the tyrosine kinases FLK1 and FLT1. Examples of VEGF receptor agonists suitable for use with the present invention include adenosine and anti-idiotypic antibodies.

The methods of the present invention can be employed under any circumstances and to any site where a subject is in need or desirous of hair growth as mentioned above. Among such instances include injury or damage to hair follicles, surgical trauma, bum wound, radiation or chemotherapy treatment site, incisional wound, donor site wound from skin transplant, ulcer, alopecia, or in order to modify physical appearance. The inventive VEGF complexes can also be administered in order to enhance one or more of the following hair qualities: brilliance, fullness, gloss, glow, length, luster, patina sheen, shine, thickness and volume.

Typically, the methods and compositions described in this aspect of the present invention comprise an effective amount of VEGF, VEGF derivative, fragment, or receptor agonist, endogenous VEGF receptor agonist agent, prodrug form of the foregoing or salt form thereof, in a non-covalent association complex with a positively-charged backbone having a plurality of attached efficiency groups, that is effective to induce or stimulate new hair growth, increase hair growth or prevent hair regression in a mammalian subject. While the compositions of the present invention can be of use with any mammalian subject in need of hair growth, the inventive compositions are especially contemplated for use with the following: humans, monkeys, cats, cows, dogs, gerbils, goats, guinea pigs, hamsters, horses, mice, prairie dogs, rabbits, rats, sheep and squirrels. In a preferred embodiment, the methods of the present invention are used with humans.

The methods of the present invention are useful for treating mammalian subjects under a variety of circumstances. For instance, the methods of the present invention can be used when the subject has a condition selected from the group consisting of alopecia, accidental injury, damage to hair follicles, surgical trauma, burn wound, radiation or chemotherapy treatment site, incisional wound, donor site wound from skin transplant, or ulcer. The methods of the present invention contemplate administering the inventive VEGF complexes in different physical forms, examples of which include creams and ointments, electroporation formulations, foams, gels, liquids, including solutions, suspensions or emulsions, lotions, microspheres and other degradable microbeads, muds, oils and pastes, ointments, patches, powders, roller sticks, salves, soaps and surfactants, sprays, etc. Gels that are suitable for use in the present invention include those described in U.S. Pat. No. 6,333,057 at col. 6, lines 6-19, which passage is incorporated by reference herein. Microspheres that are suitable for use with the present invention can comprise controlled release microspheres or microbeads, although capsule- and disk-shaped devices can also be used. The foregoing micro devices can be comprised of biodegradable or bioerodable polymers, examples of which include poly (lactic-co-glycolides) (PLGAs), polyethylene glycols (PEGs), polyanhydrides and polyorthoesters, etc. As release methods are directly related to polymer degradation, the optimal micro device shape for a given polymer will depend upon its degradation mechanism, as will be understood by those knowledgeable in the relevant art. According to one embodiment, the hair growth inducing compositions of the present invention are provided in a topical moisturizer base that comprises microspheres.

According to the methods of the present invention, the VEGF hair growth inducing medicaments can be administered to a subject utilizing any of a number of different methods, examples of which include topical application or by injection. When the compositions of the present invention are desired for subcutaneous administration, they can be provided in solution form for injection by needle. Administration can be via single injection or take place throughout a course of multiple injections. In one embodiment, the compositions of the present invention are provided in sterile form for subcutaneous injection. According to a preferred embodiment, the compositions of the present invention are applied topically.

Compositions

The present invention also provides compositions comprising a pharmaceutically or cosmeceutically acceptable carrier and an effective amount of vascular endothelial growth factor (VEGF), VEGF derivatives, VEGF fragments, VEGF receptor agonists, prodrug forms of the foregoing or salt forms thereof in a non-covalent association complex with a positively-charged backbone having a plurality of attached efficiency groups, the amount being effective for inducing or stimulating new hair growth, increasing hair growth or preventing hair regression in a mammalian subject. The positively-charged backbone comprises a polymer having attached positively-charged branching groups. According to a preferred embodiment, the compositions of the present invention comprise VEGF or VEGF receptor agonists in concentrations that are from about 1.0×10⁻³⁰ M to about 1.0×10⁴ M; preferably from about 1.0×10⁻²⁸ M to about 1.0×10³ M and most preferably from about 1×10⁻26 M to about 1.0×10²M. According to a preferred embodiment of the invention, the inventive VEGF or VEGF receptor agonist compositions are applied topically. For topical applications, the concentration of VEGF or VEGF receptor agonist is from about 1.0×10⁻25 M to about 10.0 M, preferably from about 1.0×10⁻22 M to about 1.0 M and most preferably from about 1.0×10⁻¹⁸ M to about 0.1 M. The ranges provided above illustrate ranges that are generally applicable across the entire class of active agents. Depending on the agent used, the formulation and method of administration, ranges will often be selected by the skilled practitioner from, for example, 1.0×10⁻²⁵ M to about 1.0×10⁻²² M, 1.0×10⁻²² M to about 1.0×10⁻¹⁹ M, 1.0×10⁻¹⁹M to about 1.0×10⁻¹⁶ M, 1.0×10⁻¹⁶ M to about 1.0×10⁻¹³ M, 1.0×10⁻¹³ M to about 1.0×10⁻¹⁰ M, 1.0×10⁻¹⁰ M to about 1.0×10⁻⁷ M, 1.0×10⁻⁷ M to about 1.0×10⁻⁴ M, and 1.0×10⁻⁴ M to about 1.0×10⁻¹ M.

The positively-charged backbone is typically a linear chain of atoms, either with groups in the chain carrying a positive charge at physiological pH, or with groups carrying a positive charge attached to side chains extending from the backbone. The linear backbone is a hydrocarbon backbone which is, in some embodiments, interrupted by heteroatoms selected from nitrogen, oxygen, sulfur, silicon and phosphorus. The majority of backbone chain atoms are usually carbon atoms. Additionally, the backbone will often be a polymer of repeating units (e.g., amino acids, poly(ethyleneoxy), poly(propyleneamine), and the like). In one group of embodiments, the positively-charged backbone is a polypropyleneamine wherein a number of the amine nitrogen atoms are present as ammonium groups (tetra-substituted) carrying a positive charge. In another group of embodiments, the backbone has attached a plurality of side chain moieties that include positively charged groups (e.g., ammonium groups, pyridinium groups, phosphonium groups, sulfonium groups, guanidinium groups, or amidinium groups). The side chain moieties in this group of embodiments can be placed at spacings along the backbone that are consistent or variable in separation. Additionally, the length of the side chains can be similar or dissimilar. For example, in one group of embodiments, the side chains can be linear or branched hydrocarbon chains having from one to twenty carbon atoms and terminating at the distal end (away from the backbone) in one of the above-noted positively-charged groups.

In one group of embodiments, the positively-charged backbone is a polypeptide having multiple positively charged side chain groups (e.g., lysine, arginine, ornithine, homoarginine, and the like). One of skill in the art will appreciate that when amino acids are used in this portion of the invention, the side chains can have either the D- or L-form (R or S configuration) at the center of attachment.

Alternatively, the backbone can be an analog of a polypeptide such as a peptoid. See, for example, Kessler, Angew. Chem. Int. Ed. Engl. 32:543 (1993); Zuckermann, et al., Chemtracts-Macromol. Chem. 4:80 (1992); and Simon, et al., Proc. Nat'l. Acad. Sci. USA 89:9367 (1992). Briefly, a peptoid is a polyglycine in which the side chain is attached to the backbone nitrogen atoms rather than the α-carbon atoms. As above, a portion of the side chains will typically terminate in a positively charged group to provide a positively charged backbone component. Synthesis of peptoids is described in, for example, U.S. Pat. No. 5,877,278. As the term is used herein, positively charged backbones that have a peptoid backbone construction are considered “non-peptide” as they are not composed of amino acids having naturally occurring side chains at the α-carbon locations.

A variety of other backbones can be used employing, for example, steric or electronic mimics of polypeptides wherein the amide linkages of the peptide are replaced with surrogates such as ester linkages, thioamides (—CSNH—), reversed thioamide (—NHCS—), aminomethylene (—NHCH₂—) or the reversed methyleneamino (—CH₂NH—) groups, keto-methylene (—COCH₂—) groups, phosphinate (—PO₂RCH₂—), phosphonamidate and phosphonamidate ester (—PO₂RNH—), reverse peptide (—NHCO—), trans-alkene (—CR═CH—), fluoroalkene (—CF═CH—), dimethylene (—CH₂CH₂—), thioether (—CH₂S—), hydroxyethylene (—CH(OH)CH₂—), methyleneoxy (—CH₂O—), tetrazole (CN₄), sulfonamido (—SO₂NH—), methylenesulfonamido (—CHRSO₂NH—), reversed sulfonamide (—NHSO₂—), and backbones with malonate and/or gem-diamino-alkyl subunits, for example, as reviewed by Fletcher, et al., ((1998) Chem. Rev. 98:763) and detailed by references cited therein. Many of the foregoing substitutions result in approximately isosteric polymer backbones relative to backbones formed from α-amino acids.

In each of the backbones provided above, side chain groups can be appended that carry a positively charged group. For example, the sulfonamide-linked backbones (—SO₂NH— and —NHSO₂—) can have side chain groups attached to the nitrogen atoms. Similarly, the hydroxyethylene (—CH(OH)CH₂—) linkage can bear a side chain group attached to the hydroxy substituent. One of skill in the art can readily adapt the other linkage chemistries to provide positively charged side chain groups using standard synthetic methods.

In a particularly preferred embodiment, the positively charged backbone is a polypeptide having branching groups (also referred to as efficiency groups) comprising—(glyₙ₁-(arg)ₙ₂, HIV-TAT or fragments thereof, in which the subscript n1 is an integer of from 0 to 20, more preferably 0 to 8, still more preferably 2 to 5, and the subscript n2 is an odd integer of from about 5 to about 25, more preferably about 7 to about 17, most preferably about 7 to about 13. Still further preferred are those embodiments in which the HIV-TAT fragment has the formula (gly)ₚ-RGRDDRRQRRR-(gly)_{q} or (gly)ₚ-YGRKKRRQRRR-(gly)_{q} wherein the subscripts p and q are each independently an integer of from 0 to 20 and the fragment is attached to the backbone via either the C-terminus or the N-terminus of the fragment. Preferred HIV-TAT fragments are those in which the subscripts p and q are each independently integers of from 0 to 8, more preferably 2 to 5.

In another particularly preferred embodiment, the backbone portion is a polylysine and positively charged branching groups are attached to the lysine side chain amino groups. The polylysine used in this particularly preferred embodiment can be any of the commercially available polylysines such as, for example, polylysine having MW>70,000, polylysine having MW of 70,000 to 150,000, polylysine having MW 150,000 to 300,000 and polylysine having MW>300,000 (available, for example, from Sigma Chemical Company, St. Louis, Mo., USA). The appropriate selection of a polylysine will depend on the remaining components of the composition and will be sufficient to provide an overall net positive charge to the composition and provide a length that is preferably from one to four times the combined length of the negatively charged components. Preferred positively charged branching groups or efficiency groups include, for example,—gly-gly-gly-arg-arg-arg-arg-arg-arg-arg (-gly₃arg₇) or HIV-TAT. According to one embodiment, the degree of saturation of -gly₃arg₇ branching groups is from about 5% to about 30% (i.e., from about 5 to about 30 of each 100 lysine residues is conjugated to a -gly₃arg₇). According to a more preferred embodiment, the degree of saturation of -gly₃arg₇ branching groups is from about 10% to about 25%.

In another embodiment the positively charged backbone is included in a multi-component biological transport system, more particularly one such as is described in PCT application WO 02/007773 published Jan. 31, 2002, the entire contents of which are hereby incorporated herein. That system comprises a non-covalent association complex that includes

a) a positively-charged backbone (as described above);

b) a VEGF compound; and

c) at least one member selected from:

i) a first negatively-charged backbone having one or more attached imaging moieties;

ii) a second negatively-charged backbone having one or more attached therapeutic moieties

iii) at least one member selected from RNA, DNA, ribozymes, modified oligonucleotides and CDNA encoding a selected transgene; and

iv) DNA encoding at least one persistence factor; wherein the association complex carries a net positive charge.

Compositions according to this invention may comprise only a positively-charged backbone containing enhanced efficiency groups and a VEGF compound. Such compositions will generally be in the form of a dry solid such as a powder or the like. However, the compositions of the invention may also comprise a pharmaceutically or cosmeceutically acceptable carrier. The term “cosmeceutical” as used herein relates to a cosmetic or aesthetic quality, parameter or attribute, etc., as contrasted with a pharmaceutical or clinical quality, parameter or attribute, respectively. Accordingly, a cosmeceutical composition is one that can be utilized for purposes of cosmetic or aesthetic enhancement. Cosmeceutical purposes are those that are desired and not used solely because of clinical or prophylactic indications. In a preferred embodiment of the present invention, cosmeceutical compositions are those useful for skin or body care.

A pharmaceutically or cosmeceutically acceptable carrier according to the present invention comprises at least one member selected from among antimicrobials, moisturizers and hydration agents, penetration agents, preservatives, viscosity-controlling agents and water. The term “antimicrobial” as used herein refers to a drug, antibiotic agent, etc., that is inimical to microbes. Antimicrobials include antifungals and antibacterials or bacteriostatic agents. Bacteriostatic agents can prevent bacterial degradation of the compositions upon storage. Examples of suitable bacteriostats can be found, for example, in the '572 patent to Unger, et al., supra, at col. 26, lines 55 to 62, which passage is incorporated herein by reference. As used herein, the term “penetrating agent” refers to a substance that promotes increased penetration of a substance into the skin. According to one embodiment of the present invention, examples of suitable carriers include: Cetaphil® (Galderma Laboratories, L. P., Ft. Worth, Tex.), Lac-Hydring (Bristol-Meyers Squibb), Norwegian Formula® Body Lotion (Neutrogena) and Vaseline Intensive Care® Lotion (Unilever), as well as many other commercially-available skin care products.

The negatively-charged backbones used to carry the imaging moieties, targeting moieties, and/or other therapeutic agents can be a variety of backbones having multiple groups carrying a negative charge at physiological pH. Suitable negatively-charged groups are carboxylic acids, phosphinic, phosphonic or phosphoric acids, sulfinic or sulfonic acids, and the like. In some embodiments, the negatively-charged backbone will be an oligonucleic acid. In other embodiments, the negatively-charged backbone is an oligosaccharide (e.g., dextran). In still other embodiments, the negatively-charged backbone is a polypeptide (e.g., poly glutamic acid, poly aspartic acid, or a polypeptide in which glutamic acid or aspartic acid residues are interrupted by uncharged amino acids). The moieties described in more detail below (imaging moieties, targeting agents, and therapeutic agents) can be attached to a backbone having these pendent groups, typically via ester linkages. Alternatively, amino acids which interrupt negatively-charged amino acids or are appended to the terminus of the negatively-charged backbone, can be used to attach imaging moieties and targeting moieties via, for example, disulfide linkages (through a cysteine residue), amide linkages, ether linkages (through serine or threonine hydroxyl groups) and the like.

Imaging Moieties

A variety of diagnostic or imaging moieties are useful in the present invention and are present in an effective amount that will depend on the condition being diagnosed or imaged, the route of administration, the sensitivity of the agent and device used for detection of the agent, and the like.

Examples of suitable imaging or diagnostic agents include radiopaque contrast agents, paramagnetic contrast agents, superparamagnetic contrast agents, CT contrast agents and other contrast agents. For example, radiopaque contrast agents (for X-ray imaging) will include inorganic and organic iodine compounds (e.g., diatrizoate), radiopaque metals and their salts (e.g., silver, gold, platinum and the like) and other radiopaque compounds (e.g., calcium salts, barium salts such as barium sulfate, tantalum and tantalum oxide). Suitable paramagnetic contrast agents (for MR imaging) include gadolinium diethylene triaminepentaacetic acid (Gd-DTPA) and its derivatives, and other gadolinium, manganese, iron, dysprosium, copper, europium, erbium, chromium, nickel and cobalt complexes, including complexes with 1,4,7,10-tetraazacyclododecane-N,N′,N″,N′″-tetraacetic acid (DOTA), ethylenediaminetetraacetic acid (EDTA), 1,4,7,10-tetraazacyclododecane-N,N′,N″-triacetic acid (DO3A), 1,4,7-triazacyclononane-N,N′,N″-triacetic acid (NOTA), 1,4,8,11-tetraazacyclotetradecane-N,N′,N″,N′″-tetraacetic acid (TETA), hydroxybenzylethylene-diamine diacetic acid (HBED) and the like. Suitable superparamagnetic contrast agents (for MR imaging) include magnetites, superparamagnetic iron oxides, monocrystalline iron oxides, particularly complexed forms of each of these agents that can be attached to a negatively charged backbone. Still other suitable imaging agents are the CT contrast agents including iodinated and noniodinated and ionic and nonionic CT contrast agents, as well as contrast agents such as spin-labels or other diagnostically effective agents.

Other examples of diagnostic agents include marker genes that encode proteins that are readily detectable when expressed in a cell, including, but not limited to, β-galactosidase, green fluorescent protein, blue fluorescent protein, luciferase, and the like. A wide variety of labels may be employed, such as radionuclides, fluors, enzymes, enzyme substrates, enzyme cofactors, enzyme inhibitors, ligands (particularly haptens), and the like. Still other useful substances are those labeled with radioactive species or components, such as ⁹⁹ mTc glucoheptonate.

Targeting Agents

A variety of targeting agents is useful in the compositions described herein. Typically, the targeting agents are attached to a negatively-charged backbone as described for the imaging moieties above. The targeting agents can be any element that makes it possible to direct the transfer of a nucleic acid, therapeutic agent or another component of the composition to a particular site. The targeting agent can be an extracellular targeting agent, which allows, for example, a nucleic acid transfer to be directed towards certain types of cells or certain desired tissues (tumor cells, liver cells, hematopoietic cells, and the like). Such an agent can also be an intracellular targeting agent, allowing a therapeutic agent to be directed towards particular cell compartments (e.g., mitochondria, nucleus, and the like).

The targeting agent or agents are preferably linked, covalently or non-covalently, to a negatively-charged backbone according to the invention. According to a preferred mode of the invention, the targeting agent is covalently attached to an oligonucleotide that serves as a negatively-charged backbone component, preferably via a linking group. Methods of attaching targeting agents (as well as other biological agents) to nucleic acids are well known to those of skill in the art using, for example, heterobifunctional linking groups (see Pierce Chemical Catalog). In one group of embodiments, the targeting agent is a fusogenic peptide for promoting cellular transfection, that is to say for favoring the passage of the composition or its various elements across membranes, or for helping in the egress from endosomes or for crossing the nuclear membrane. The targeting agent can also be a cell receptor ligand for a receptor that is present at the surface of the cell type, such as, for example, a sugar, transferrin, insulin or asialo-orosomucoid protein. Such a ligand may also be one of intracellular type, such as a nuclear location signal (nls) sequence which promotes the accumulation of transfected DNA within the nucleus.

Other targeting agents useful in the context of the invention, include sugars, peptides, hormones, vitamins, cytokines, oligonucleotides, lipids or sequences or fractions derived from these elements and which allow specific binding with their corresponding receptors. Preferably, the targeting agents are sugars and/or peptides such as antibodies or antibody fragments, cell receptor ligands or fragments thereof, receptors or receptor fragments, and the like. More preferably, the targeting agents are ligands of growth factor receptors, of cytokine receptors, or of cell lectin receptors or of adhesion protein receptors. The targeting agent can also be a sugar which makes it possible to target lectins such as the asialoglycoprotein receptors, or alternatively an antibody Fab fragment which makes it possible to target the Fc fragment receptor of immunoglobulins.

Nucleic Acids

When included in the compositions of the present invention, the nucleic acid can be either a deoxyribonucleic acid or a ribonucleic acid, and can comprise sequences of natural or artificial origin. More particularly, the nucleic acids used herein can include genomic DNA, cDNA, mRNA, tRNA, rRNA, hybrid sequences or synthetic or semi-synthetic sequences. These nucleic acids can be of human, animal, plant, bacterial, viral, etc. origin. Additionally, the nucleic acids can be obtained by any technique known to those skilled in the art, and in particular by the screening of banks, by chemical synthesis or by mixed methods including the chemical or enzymatic modification of sequences obtained by the screening of banks. Still further, the nucleic acids can be incorporated into vectors, such as plasmid vectors.

The deoxyribonucleic acids used in the present invention can be single- or double-stranded. These deoxyribonucleic acids can also code for therapeutic genes, sequences for regulating transcription or replication, antisense sequences, regions for binding to other cell components, etc. Suitable therapeutic genes are essentially any gene which codes for a protein product having a therapeutic effect. The protein product thus encoded may be a protein, polypeptide, a peptide, or the like. The protein product can, in some instances, be homologous with respect to the target cell (that is to say a product which is normally expressed in the target cell when the latter exhibits no pathology). In this manner, the use of suitable nucleic acids can increase the expression of a protein, making it possible, for example, to overcome an insufficient expression in the cell. Alternatively, the present invention provides compositions and methods for the expression of a protein which is inactive or weakly active due to a modification, or alternatively of overexpressing the protein. The therapeutic gene may thus code for a mutant of a cell protein, having increased stability, modified activity, etc. The protein product may also be heterologous with respect to the target cell. In this case, an expressed protein may, for example, make up or provide an activity which is deficient in the cell, enabling it to combat a pathology or to stimulate an immune response.

More particularly, nucleic acids useful in the present invention are those that code for enzymes, blood derivatives, hormones, lymphokines, interleukins, interferons, TNF, growth factors, neurotransmitters or their precursors or synthetic enzymes, or trophic factors: BDNF, CNTF, NGF, IGF, GMF, aFGF, bFGF, VEGF, NT3, NT5, HARP/pleiotrophin; the proteins involved in the metabolism of lipids, of apolipoprotein-types selected from apolipoproteins A-I, A-II, A-IV, B, C-I, C-II, C-III, D, E, F, G, H, J and apo(a), metabolic enzymes such as, for example, lipoprotein lipase, hepatic lipase, lecithin cholesterol acyltransferase, 7-a-cholesterol hydroxylase, phosphatidic acid phosphatase, or lipid transfer proteins such as cholesterol ester transfer protein and phospholipid transfer protein, a protein for binding HDLs or a receptor selected from, for example, LDL receptors, chylomicron-remnant receptors and scavenger receptors, dystrophin or minidystrophin, GAX protein, CFTR protein associated with mucoviscidosis, tumor-suppressant genes: p53, Rb, Rap1A, DCC, k-rev; protein factors involved in coagulation: factors VII, VIII, IX; or the nucleic acids can be those genes involved in DNA repair, suicide genes (thymidine kinase, cytosine deaminase), genes encoding thrombomodulin, α1-antitrypsin, tissue plasminogen activator, superoxide dismutase, elastase, matrix metalloproteinase, and the like.

The therapeutic genes useful in the present invention can also be an antisense sequence or a gene whose expression in the target cell makes it possible to control the expression of genes or the transcription of cellular mRNA. Such sequences can, for example, be transcribed in the target cell into complementary RNA of cellular mRNA and thus block their translation into protein, according to the technique described in patent EP 140,308. The antisense sequences also comprise the sequences coding for ribozymes which are capable of selectively destroying target RNA (see EP 321,201).

As indicated above, the nucleic acid may also contain one or more genes coding for an antigenic peptide, capable of generating an immune response in humans or animals. In this particular embodiment, the invention thus makes it possible to produce either vaccines or immunotherapeutic treatments applied to humans or to animals, in particular against microorganisms, viruses or cancers. They may in particular be antigenic peptides specific for Epstein Barr virus, for HIV virus, for hepatitis B virus (see EP 185,573), for pseudo-rabies virus or alternatively specific for tumors (see EP 259,212).

Preferably, the nucleic acid also comprises sequences that allow the expression of the therapeutic gene and/or of the gene coding for the antigenic peptide in the desired cell or organ. These can be sequences that are naturally responsible for expression of the gene considered when these sequences are capable of functioning in the infected cell. The nucleic acids can also be sequences of different origin (responsible for the expression of other proteins, or even synthetic proteins). In particular, the nucleic acids can contain promoter sequences for eukaryotic or viral genes. For example, the promoter sequences can be those derived from the genome of the cell which it is desired to infect. Similarly, the promoter sequences can be derived from the genome of a virus, e.g., the promoters of genes E1A, MLP, CMV, RSV, etc. In addition, these expression sequences may be modified by addition of activation sequences, regulation sequences, etc.

Moreover, the nucleic acid may also contain, in particular upstream of the therapeutic gene, a signal sequence which directs the therapeutic product synthesized into the secretion pathways of the target cell. This signal sequence may be the natural signal sequence of the therapeutic product, but it may also be any other functional signal sequence, or an artificial signal sequence.

DNA Encoding at Least One Persistence Factor

In some embodiments, the composition will also comprise DNA encoding at least one persistence factor. Exemplary of such DNA is the DNA encoding adenoviral preterminal protein 1 (see, Lieber, et al. Nature Biotechnology 15(13):1383-1387 (1997).

Biological Agents

A variety of biological agents, including both therapeutic and cosmeceutic agents, are useful in the present invention and are present in an effective amount that will depend on the condition being treated, prophylactically or otherwise, the route of administration, the efficacy of the agent and patient's size and susceptibility to the treatment regimen.

Suitable therapeutic agents that can be attached to a negatively charged backbone can be found in essentially any class of agents, including, for example, analgesic agents, anti-asthmatic agents, antibiotics, antidepressant agents, anti-diabetic agents, antifungal agents, antiemetics, antihypertensives, anti-impotence agents, anti-inflammatory agents, antineoplastic agents, anti-HIV agents, antiviral agents, anxiolytic agents, contraception agents, fertility agents, antithrombotic agents, prothrombotic agents, hormones, vaccines, immunosuppressive agents, vitamins and the like.

Suitable cosmeceutic agents include, for example, epidermal growth factor (EGF), as well as human growth hormone, antioxidants, and botulinum toxin (BTX).

More particularly, therapeutic agents useful in the present invention include such analgesics as lidocaine, novocaine, bupivacaine, procaine, tetracaine, benzocaine, cocaine, mepivacaine, etidocaine, proparacaine ropivacaine, prilocaine and the like; anti-asthmatic agents such as azelastine, ketotifen, traxanox, corticosteroids, cromolyn, nedocromil, albuterol, bitolterol mesylate, pirbuterol, salmeterol, terbutyline, theophylline and the like; antibiotic agents such as neomycin, streptomycin, chloramphenicol, norfloxacin, ciprofloxacin, trimethoprim, sulfamethyloxazole, the β-lactam antibiotics, tetracycline, and the like; antidepressant agents such as nefopam, oxypertine, imipramine, traza done and the like; anti-diabetic agents such as biguanidines, sulfonylureas, and the like; antiemetics and antipsychotics such as chlorpromazine, fluphenazine, perphenazine, prochlorperazine, promethazine, thiethylperazine, triflupromazine, haloperidol, scopolamine, diphenidol, trimethobenzamide, and the like; neuromuscular agents such as atracurium mivacurium, rocuronium, succinylcholine, doxacurium, tubocurarine, and botulinum toxin (BTX); antifungal agents such as amphotericin B, nystatin, candicidin, itraconazole, ketoconazole, miconazole, clotrimazole, fluconazole, ciclopirox, econazole, naftifine, terbinafine, griseofulvin and the like; antihypertensive agents such as propanolol, propafenone, oxyprenolol, nifedipine, reserpine and the like; anti-impotence agents such as nitric oxide donors and the like; anti-inflammatory agents including steroidal anti-inflammatory agents such as cortisone, hydrocortisone, dexamethasone, prednisolone, prednisone, fluazacort, and the like, as well as non-steroidal anti-inflammatory agents such as indomethacin, ibuprofen, ramifenizone, prioxicam and the like; antineoplastic agents such as adriamycin, cyclophosphamide, actinomycin, bleomycin, daunorubicin, doxorubicin, epirubicin, mitomycin, rapamycin, methotrexate, fluorouracil, carboplatin, carmustine (BCNU), cisplatin, etoposide, interferons, phenesterine, taxol (including analogs and derivatives), camptothecin and derivatives thereof, vinblastine, vincristine and the like; anti-HIV agents (e.g., antiproteolytics); antiviral agents such as amantadine, methisazone, idoxuridine, cytarabine, acyclovir, famciclovir, ganciclovir, foscamet, sorivudine, trifluridine, valacyclovir, cidofovir, didanosine, stavudine, zalcitabine, zidovudine, ribavirin, rimantatine and the like; anxiolytic agents such as dantrolene, diazepam and the like; COX-2 inhibitors; contraception agents such as progestogen and the like; anti-thrombotic agents such as GPIIb/IIIa inhibitors, tissue plasminogen activators, streptokinase, urokinase, heparin and the like; prothrombotic agents such as thrombin, factors V, VII, VIII and the like; hormones such as insulin, growth hormone, prolactin, EGF (epidermal growth factor) and the like; immunosuppressive agents such as cyclosporine, azathioprine, mizorobine, FK506, prednisone and the like; angiogenic agents; vitamins such as A, D, E, K and the like; and other therapeutically or medicinally active agents. See, for example, GOODMAN & GILMAN'S THE PHARMACOLOGICAL BASIS OF THERAPEUTICS, Ninth Ed. Hardman, et al., eds. McGraw-Hill, (1996).

Negatively-Charged Backbones Having Attached Imaging Moieties, Targeting Agents or Therapeutic Agents

For imaging moieties, targeting agents and therapeutic agents, the individual compounds are attached to a negatively charged backbone. Typically, the attachment is via a linking group used to covalently attach the particular agent to the backbone through functional groups present on the agent as well as the backbone. A variety of linking groups are useful in this aspect of the invention. See, for example, Hermanson, Bioconjugate Techniques, Academic Press, San Diego, Calif. (1996); Wong, S. S., Ed., Chemistry of Protein Conjugation and Cross-Linking, CRC Press, Inc., Boca Raton, Fla. (1991); Senter, et al., J. Org. Chem. 55:2975-78 (1990); and Koneko, et al., Bioconjugate Chem. 2:133-141 (1991).

In some embodiments, the therapeutic, diagnostic or targeting agents will not have an available functional group for attaching to a linking group, and can be first modified to incorporate, for example, a hydroxy, amino, or thiol substituent. Preferably, the substituent is provided in a non-interfering portion of the agent, and can be used to attach a linking group, and will not adversely affect the function of the agent.

In yet another aspect, the present invention provides compositions comprising a non-covalent association complex of a positively-charged backbone having at least one attached efficiency group and at least one nucleic acid member selected from the group consisting of RNA, DNA, ribozymes, modified oligonucleotides and cDNA encoding a selected transgene. In this aspect of the invention, the positively-charged backbone can be essentially any of the positively-charged backbones described above, and will also comprise (as with selected backbones above) at least one attached efficiency group. Suitable efficiency groups include, for example, (Gly)ₙ₁-(Arg)ₙ₂ (wherein the subscript n1 is an integer of from 3 to about 5, and the subscript n2 is an odd integer of from about 7 to about 17) or TAT domains. Additionally, the nucleic acids useful in this aspect of the invention are the same as have been described above.

In one preferred embodiment, the VEGF compositions of the present invention further comprise at least two members selected from the group consisting of antimicrobials, moisturizers and hydration agents, penetration agents, preservatives, viscosity-controlling agents and water. In a different preferred embodiment, the VEGF compositions of the present invention comprise at least three members selected from the group consisting of antimicrobials, moisturizers and hydration agents, penetration agents, preservatives, viscosity-controlling agents and water. In another preferred embodiment, the VEGF compositions of the present invention further comprise at least four members selected from the group consisting of antimicrobials, moisturizers and hydration agents, penetration agents, preservatives, viscosity-controlling agents and water. In a still further preferred embodiment, the VEGF compositions of the present invention further comprise at least one member selected from the group consisting of antimicrobials, moisturizers and penetration agents and at least one member selected from the group consisting of preservatives, viscosity-controlling agents and water. According to a still different preferred embodiment, the VEGF compositions of the present invention comprise at least one member selected from each of the following: antimicrobials, moisturizers, preservatives and water. According to still another preferred embodiment, the VEGF compositions of the present invention further comprise at least one moisturizer and at least one preservative.

In addition to the pharmaceutically or cosmeceutically acceptable carriers mentioned above, the VEGF, VEGF receptor agonists, prodrug forms thereof or salts of the foregoing and the positively-charged backbone discussed earlier, the medicaments of the present invention can also include optional components. Optional components that are suitable for use with the medicaments of the present invention include one or more of the following: anesthetics, anti-itch actives, botanical extracts, color agents, conditioning agents, darkening or lightening agents, fragrance, glitter, hair pigment additives, humectants, mica, minerals, oils, polyphenols, silicones or derivatives thereof, sunblocks, surfactants, vitamins, waxes, and phytomedicinals. As used herein, the term “humectant” is understood to refer to any first substance that is added to any second substance in order to keep it moist. Mica can be used in the VEGF compositions of the present invention to enhance hair shine or to add glitter to hair, and polyphenols can function as antioxidants. Silicones or silicone derivatives that are suitable for use with the present invention comprise film-forming agents, emollients, shine enhancers, smoothing agents, or agents that provide a decrease in oily hand feel.

According to a preferred embodiment, the medicaments of the present invention optionally include at least one component selected from among anesthetics, anti-itch actives, botanical extracts, humectants, silicones or derivatives thereof and phytomedicinals, at least one component selected from among conditioning agents, darkening or lightening agents, hair pigment additives, minerals, polyphenols and sunblocks, and at least one component selected from among fragrance, glitter, mica, surfactants, vitamins, and waxes. According to a more preferred embodiment, the medicaments of the present invention optionally include at least one component selected from among anesthetics, anti-itch actives and botanical extracts, at least one component selected from among conditioning agents, darkening or lightening agents and hair pigment additives, and at least one component selected from among fragrance, surfactants and vitamins. According to a most preferred embodiment, the medicaments of the present invention optionally include at least one of each of the following: botanical extracts, conditioning agents and vitamins.

The medicaments of the present invention are suitable for dispensing from a number of containers, examples of which include bottles, brushes, cans, combs, controlled-release matrices, fabrics, pumps, sprayers, especially aerosol spray dispensers, self-pressurized spray dispensers and non-aerosol spray dispensers, tubes, vials, and wands. As used herein, the term “controlled-release matrices” refer to those that release an active component substantially continuously over a variable period of time.

The compositions and methods described herein are particularly suitable for the promotion of hair growth and the prevention or treatment of hair regression. In view of the fact that VEGF receptor agonists can induce hair growth, however, in another aspect of the present invention, VEGF receptor antagonists can be used to induce or promote hair regression. Thus, it is also within the scope of the present invention that in certain instances where a limit in the length of hair growth or reduction in the number of hair follicles is desired, the present invention also provides methods for inducing or stimulating hair regression, limiting hair growth or preventing hair growth in a mammalian subject. The method comprises administering a pharmaceutically or cosmeceutically effective amount of a composition comprising a VEGF receptor antagonist, a prodrug form thereof or a salt form of the foregoing to the subject. Examples of VEGF receptor antagonists suitable for use with the present invention include the peptide TWLPPR, or human prolactin (or the 16 kD n-terminal fragment thereof).

The present invention also includes applications of VEGF, VEGF receptor agonists or endogenous VEGF receptor agonist agents for fat stabilization. Fat stabilization, particularly in humans, is generally associated with the appearance of aging attributed to fat atrophy as well as fat regression in the skin. The VEGF methods and compositions described herein can assist in preventing the formation of wrinkles and aid in ameliorating the appearance of deep wrinkles by supporting vascularity of the skin.

Uses

In a further aspect, the present invention provides the use of vascular endothelial growth factor (VEGF), a VEGF receptor agonist, a prodrug form of the foregoing or a salt form thereof in non-covalent association with a complex of a positively-charged backbone having a plurality of attached efficiency groups in the preparation of a medicament for topical application to stimulate new hair growth, increase hair growth or prevent hair regression in a mammalian subject Typical medicaments are those that comprise a cosmeceutically or pharmaceutically acceptable carrier in addition to the VEGF compound and positively-charged backbone having a plurality of attached efficiency groups just mentioned.

Kits

In another aspect, the present invention provides a kit for inducing or stimulating new hair growth, increasing hair growth or preventing hair regression in a mammalian subject. The kit of the present invention comprises a composition and a container, wherein the composition comprises a pharmaceutically or cosmeceutically acceptable carrier and an effective amount of vascular endothelial growth factor (VEGF), a VEGF receptor agonist, a prodrug form of the foregoing or a salt form thereof in non-covalent association with a complex of a positively-charged backbone having a plurality of attached efficiency groups. Different dispensing containers are contemplated for use with the compositions of the present invention, examples of which include bottles, brushes, cans, combs, controlled-release matrices, fabrics pumps, sprayers, especially aerosol spray dispensers, self-pressurized spray dispensers and non-aerosol spray dispensers, tubes, vials, and wands. As used herein, the term “controlled-release matrices” refer to those that release an active component substantially continuously over a variable period of time.

The kits of the present invention can contain hair growth compositions in matrices that provides for the release of pharmaceutical or cosmeceutical medicaments over a course of time of from one to twenty-four hours. Alternately, the kits of the present invention can deliver hair growth compositions in matrices that provide for the release of pharmaceutical or cosmeceutical medicaments over a course of time of from one to twenty eight days. According to a preferred embodiment of the invention, the inventive VEGF medicaments described herein are delivered substantially continuously from a matrix over the course of a day. In another preferred embodiment of the invention, the inventive hair growth compositions are delivered substantially continuously from a matrix over an eight hour period. According to another embodiment of the present invention, the inventive VEGF compositions are delivered in a matrix that is not readily washed off or removed with water. As used herein, the phrase “not readily washed off or removed with water” is meant to indicate that the matrices that are contemplated for use with the medicaments of the present invention maintain their contact with a subject despite incidental or accidental contact with water.

According to one embodiment, the hair growth compositions of the present invention can be delivered with cosmeceuticals such as those used in eye pencils and pens; with brushes and vials as in eye liners and mascaras; in the form of masques and mud packs; in shampoos and conditioners, etc. Fabrics that are contemplated for use in delivering the hair inducing compositions of the present invention include those which can be worn on a portion of the body. Examples of such dispensing containers include caps which can be worn while sleeping or convalescing, bandages that can be wrapped or attached to injury sites, etc.

### EXAMPLES

### Example 1

A first experiment was conducted in order to evaluate the therapeutic benefit of the complexes of the present invention on the promotion of hair growth. Based on a series of preliminary studies, it was determined that dosages of about 0.002 mg (2 μg) vascular endothelial growth factor per kilogram body weight were desirable. Accordingly, 720 μl of a 10 μg/ml stock solution of VEGF (VEGF₁₆₅ (>97%), from Calbiochem®, San Diego, Calif.) was prepared for all experiments. Treatment solutions were prepared as described below.

Solution A. Competitive transfection solution: 0.08 ml (80 μl) sterile deionized water (DI) were combined with 0.16 ml (160 μl) of a positively-charged polypeptide backbone that lacked attached efficiency groups (polylysine, available from Sigma-Aldrich Corp., St. Louis, Mo.) and 0.08 ml (80 μl) of the VEGF stock solution above.

Solution B. KNR transfection solution: 0.08 ml (80 μl) sterile deionized water (DI) were combined with 0.16 ml (160 μl) of a positively-charged polypeptide backbone that contained a plurality of attached high efficiency groups (KNR) and 0.08 ml (80 μl) of the VEGF stock solution above. The KNR is a polylysine backbone that contains efficiency groups (gly)_{3 (arg)}₇ attached to side chains of the polylysine backbone. The degree of (gly)₃(arg)₇ saturation for the KNR used was about 15%.

Solution C. Control solution: 0.24 ml (240 μl) sterile deionized water (DI) were combined with 0.08 ml (80 μl) of the VEGF stock solution described above. There was no positively-charged polypeptide backbone present in Solution C.

Method. To a 0.2 ml aliquot of a moisturizer carrier (Cetaphil®, supra), were added 0.02 ml (20 μl) of Solution A, B or C to generate samples labeled Test Solutions A, B, or C, respectively. After the foregoing additions were performed, the solutions were mixed to homogeneity and stored at 4° C. overnight. Test Solution A contained a positively-charged polypeptide backbone that lacked attached efficiency groups, and thus represented one competitive test solution. Test Solution B contained a positively-charged polypeptide backbone with attached high efficiency groups, and Test Solution C was a control that contained VEGF with neither positively-charged backbone nor attached efficiency groups.

Six black mice (C57) at eight weeks of age were used as test subjects (e.g. JAX® mice available from Jackson Laboratories, Bar Harbor, Me.). The mice were anesthetized with 3% isoflurane by inhalation, shaved, and underwent depilation at mid-scapular dorsal region of 2 cm×2 cm with a rosin mixture (Ardell Surgi-Wax™ from American International Industries, City of Commerce, CA). The depilation was performed in order to induce synchronized growth of an adolescent first hair cycle in the subjects.

Throughout a fourteen day testing period, approximately 0.2 ml aliquots of Test Solutions A, B or C were applied daily to the mice test subjects in each group. The testing groups and their treatment regiment consisted of the following:

1) Group A, the comparison group, consisted of two mice, which were treated with Test Solution A, an example of a competitive test solution;

2) Group B consisted of two mice, which were treated with Test Solution B, a VEGF medicament according to the present invention; and

3) Group C, the control group, consisted of two mice, which treated with Test Solution C, a control test solution.

After 14 days application, the treated skin segments from each test subject were harvested en bloc and subdivided into three equal portions: a cranial portion, a left lateral portion and a right lateral portion. The cranial portions and the left lateral portions were fixed in 10% neutral buffered formalin for 12-16 hours, then rinsed in 70% ethanol and embedded in paraffin. The right lateral portions were snap frozen in optimal cutting temperature (OCT) medium at the time of harvest and promptly stored at −35° C. for later use. The paraffin-embedded specimens were sectioned at 4-6 microns, deparaffinized, and stained with a combination of Verhoeff elastica-Masson trichrome stain for morphological assessment of follicle area and number. (Verhoeff elastica stain is available, for example, from Newcomer Supply Middleton, Wis.; Masson trichrome stain is available, for example, from Energy Beam Sciences, Inc., Agawam, Mass.)

Frozen samples underwent random hair pulls to determine hair shaft length. All procedures and analyses were performed by observers under blind test conditions. High resolution digital micrographs of each preparation were obtained using a Diagnostic Instruments SPOT camera (Diagnostic Instruments, Sterling Heights, Mich.) as displayed on a Nikon E600 epifluorescence microscope with plan apochromat lenses. Images were analyzed using Image Pro® Plus software (Media Cybernetics, Silver Spring, Md.) to permit determinations of total cross-sectional follicle area, follicle number per follicle area, number of follicles without a hair shaft (i.e., follicles that do not recover from injury) and hair length. Mean and standard errors were assessed using Statview (Abacus Concepts, Berkeley, Calif.), with comparisons made using ANOVA repeated measurements and significance determined at 95% with post-hoc testing using Fisher protected least significant difference (PLSD) or Scheffe F-tests. The results which were obtained are provided in tabular form below.

Experiment 1. In a first experiment, hair shaft lengths were measured for each of the test samples. Table 1 provides hair shaft lengths in units of pixels (where 1 pixel equals 2.774 microns) for the samples from Group A, Group B and Group C.

TABLE 1
Hair Shaft Length (in Pixels)

| Group: | Mean: | Std. Error: |
|---|---|---|
| A | 1170.248 | 0.955 |
| B | 1664.067 | 89.791 |
| C | 1131.009 | 60.440 |

| | | ANOVA | ANOVA |
|---|---|---|---|
| Comparison: | individual p value | (95%) | (99%) |
| A vs. B | *0.0363 | ** | ** |
| A vs. C | 0.8283 | | |
| B vs. C | *0.0001 | ** | ** |

* = significant by Fisher PLSD,
** = significant by Fisher and Scheffe

As can be seen from the results of Experiment 1 shown in Table 1 above, the mice treated with Test Solution A in Group A showed little better than a 3% (nonsignificant) improvement in hair shaft length as compared to the control mice in Group C after fourteen days. However, the mice treated with the Test Solution B in Group B exhibited better than a 47% (statistically significant) increase in hair shaft length as compared to the mice treated with control Test Solution C in Group C.

Experiment 2. In a second experiment, the number of follicles that do not recover per unit area were counted for each of the test samples. Table 2 provides numbers of non-recovering hair follicles as a percentage of the total number of hair follicles examined for the samples from each of Groups A, B and C.

TABLE 2
Follicles That Do Not Recover After Fourteen (14) Days (%)

| Group: | Mean: | Std. Error: |
|---|---|---|
| A | 3.601 | 0.622 |
| B | 1.801 | 0.577 |
| C | 3.370 | 0.821 |

| Comparison: | individual p value | ANOVA (95%) |
|---|---|---|
| A vs. B | P = 0.0229 | * |
| A vs. C | P > 0.05 | |
| B vs. C | P = 0.044 | * |

* = significant by Fisher PLSD,
** = significant by Fisher and Scheffe

The results from Experiment 2 in Table 2 above show that approximately 3.6% of the follicles from the mice of Group A that were treated with competitive Test Solution A did not recover after treatment according to Experiment 1 above, and less than 3.4% of the follicles from the mice of control Group C that were treated with Test Solution C did not recover. By contrast, almost half that number, 1.8%, of the follicles from the mice of Group B that were treated with the inventive compositions in Test Solution B did not recover. That is, there was nearly a two to one statistically significant reduction in the number of hair follicles that were unable to form a new hair shaft following exposure to the inventive compositions (Test Solution B) as compared to either the competitive solution (Test Solution A) or the control solution (Test Solution C).

Experiment 3. In addition to hair shaft length and the number of non-recovering follicles, follicle areas were also measured for hair samples taken from the mice in Example 1. As part of Experiment 3, the data presented in Table 3 below provides follicle area measurements of the cross-sectional area in units of square pixels (1 square pixel equals 7.69 square microns) for the samples from Groups A, B and C.

TABLE 3
Total Follicle Cross-Sectional Area (in Square Pixels)

| Group: | Mean: | Std. Error: |
|---|---|---|
| A | 32774.125 | 5748.063 |
| B | 57582.500 | 3219.590 |
| C | 32458.333 | 2477.525 |

| Comparison: | ANOVA (95%) |
|---|---|
| A vs. B | ** |
| A vs. C | |
| B vs. C | ** |

* = significant by Fisher PLSD,
** = significant by Fisher and Scheffe

As can be seen from the results of Experiment 3 shown in Table 3 above, the mice treated with Test Solution A in Group A exhibited not quite a 1% improvement in follicle cross-sectional area as compared to the control mice in Group C after fourteen days. However, the mice in Group B that were treated with Test Solution B exhibited a 177-fold increase, or an improvement of over 77%, in follicle cross-sectional area as compared to the mice of Group C after the same fourteen day period.

Experiment 4. In a fourth experiment, mean follicle cross-sectional areas were determined for hairs taken from the mice in Example 1. As part of Experiment 4, the data presented in Table 4 below provides mean follicle cross-sectional area in square pixels (1 square pixel equals 7.69 square microns) for samples from mice in Group A treated with Test Solution A, mice form Group B treated with Test Solution B and mice in Group C treated with Test Solution C.

TABLE 4
Mean Follicle Cross-Sectional Area (in Square Pixels)

| Group: | Mean: | Std. Error: |
|---|---|---|
| A | 103.073 | 6.841 |
| B | 145.637 | 4.134 |
| C | 120.339 | 7.387 |

| Comparison: | ANOVA (95%) |
|---|---|
| A vs. B | ** |
| A vs. C | |
| B vs. C | ** |

* = significant by Fisher PLSD,
** = significant by Fisher and Scheffe

As can be seen from the results shown in Table 4 above, the mice treated with Test Solution A in Group A actually showed an overall decrease of about 14% in cross-sectional area as compared to the control mice in Group C after fourteen days. However, the mice of Group B treated with the inventive medicaments of the present invention in Test Solution B exhibited better than a 21% increase in mean follicle cross-sectional area as compared to the control mice in Group C. These results represent an increase in mean follicle cross-sectional area of nearly 42% for the mice of Group B as compared to the mice of Group A that were treated with competitor solution A.

Experiment 5. In a fifth experiment, the number of follicles per cross section was determined for over standardized length cross-sections of skin taken from the mice in Example 1. As part of Experiment 5, the data presented in Table 5 below provides numbers of follicles per cross section area for hair samples obtained from mice in Group A treated with Test Solution A, mice from Group B treated with Test Solution B and mice in Group C treated with Test Solution C.

TABLE 5
Number of Follicles

| Group: | Mean: | Std. Error: |
|---|---|---|
| A | 304.38 | 28.676 |
| B | 396.75 | 15.422 |
| C | 304.5 | 22.626 |

| Comparison: | ANOVA (95%) |
|---|---|
| A vs. B | * |
| A vs. C | |
| B vs. C | * |

* = significant by Fisher PLSD,
** = significant by Fisher and Scheffe

The results for Experiment 5 provided in Table 5 above show that there was less than a 0.03% change in number of follicles per cross section area for hairs taken from the mice of Group A treated with Solution A as compared to hairs from mice of Group C treated with Solution C. By contrast, the mice of Group B treated with the inventive solutions of Test Solution B exhibited over a 30% increase in follicle number as compared to the Group C control group.

Overall, the results obtained for Experiments 1-5 above show that compositions containing VEGF with no transdermal delivery platform do not, in general, perform as well as compositions that do contain a non-covalently associated transdermal delivery platform. Compositions with a weak transdermal delivery platform tend to induce improved hair qualities albeit nonsignificantly over compositions lacking a polymer transdermal platform, while compositions having a transdermal delivery platform that includes attached efficiency groups provides the most improved hair qualities. That these enhancements in hair growth are valid are seen in that the results obtained among the different testing groups are statistically significant.

Although the foregoing invention has been described in some detail by way of illustration and example for purposes of clarity of understanding, it will be apparent to those skilled in the art that various changes and modifications can be practiced without departing from the spirit of the invention. Therefore, the foregoing descriptions and examples should not be construed as limiting the scope of the invention, and are to be included within the spirit and purview of this application and of the appended claims.

What is claimed is:

1. A method for inducing or stimulating new hair growth, increasing hair growth or preventing hair regression in a mammalian subject in need of or desirous of such treatment, comprising administering to the subject a pharmaceutically or cosmeceutically effective amount of a composition comprising vascular endothelial growth factor (VEGF), a VEGF receptor agonist, a prodrug form of the foregoing or a salt form thereof in a non-covalent association complex with a positively-charged backbone having a plurality of attached efficiency groups.

2. The method of claim 1, wherein the positively-charged backbone comprises a polymer having attached positively-charged branching groups.

3. The method of claim 1, wherein the positively-charged backbone is polylysine.

4. The method of claim 1, wherein the efficiency group is selected from the group consisting of (gly)ₙ₁(arg)ₙ₂, wherein the subscript nil is an integer of from about 2 to about 5, and the subscript n2 is an odd integer of from about 7 to about 17, and TAT domains or fragments thereof.

5. The method of claim 1, wherein the positively-charged backbone having a plurality of efficiency groups is a 150,000 to 300,000 MW polylysine backbone having a plurality of attached gly₃arg₇ groups wherein the degree of lysine saturation is from 5% to about 30%

6. The method of claim 1, wherein the composition further comprises
at least one member selected from:
i) a first negatively-charged backbone having one or more attached imaging moieties;
ii) a second negatively-charged backbone having one or more attached therapeutic moieties
iii) at least one member selected from RNA, DNA, ribozymes, modified oligonucleotides and cDNA encoding a selected transgene; and
iv) DNA encoding at least one persistence factor;
wherein the association complex carries a net positive charge.

7. The method of claim 1, wherein the composition further comprises at least one member selected from the group consisting of antimicrobials, moisturizers and hydration agents, penetration agents, preservatives, viscosity-controlling agents and water, and optionally including anesthetics, anti-itch actives, botanical extracts, color agents, conditioning agents, darkening or lightening agents, fragrance, glitter, hair pigment additives, humectants, mica, minerals, oils, polyphenols, silicones or derivatives thereof, sunblocks, surfactants, vitamins, waxes, and phytomedicinals.

8. The method of claim 1, wherein the composition further comprises a pharmaceutically or cosmeceutically acceptable carrier.

9. The method of claim 1, wherein the composition is in the form of creams and ointments, electroporation formulations, foams, gels, liquids including solutions, suspensions or emulsions, lotions, microspheres and other degradable microbeads, muds, oils and pastes, ointments, patches, powders, roller sticks, salves, soaps and surfactants, sprays.

10. The method of claim 9, wherein the carrier is a moisturizer and the composition is in the form of microspheres.

11. The method of claim 10, wherein the microspheres are comprised of a polymer selected from the group consisting of poly(lactic-co-glycosides), polyethylene glycols, polyanhydrides and polyorthoesters.

12. The method of claim 1, wherein the administering is subcutaneous or topical.

13. The method of claim 1, wherein the administering is topical.

14. The method of claim 1, wherein the mammalian subject is selected from the group consisting of humans, monkeys, cats, cows, dogs, gerbils, goats, guinea pigs, hamsters, horses, mice, prairie dogs, rabbits, rats, sheep and squirrels.

15. The method of claim 1, wherein the mammalian subject is a human.

16. The method of claim 1, wherein the mammalian subject has a condition selected from the group consisting of alopecia, accidental injury, damage to hair follicles, surgical trauma, burn wound, radiation or chemotherapy treatment site, incisional wound, donor site wound from skin transplant and ulcer; or a desire to modify physical appearance.

17. The method of claim 1, wherein administering the composition to the subject enhances one or more hair properties selected from the group consisting of brilliance, fullness, gloss, glow, length, luster, patina, sheen, shine, thickness and volume.

18. The method of claim 13, wherein the composition comprises VEGF in a concentration of from about 1×10⁻²⁵ M to about 10.0 M.

19. A composition comprising a pharmaceutically or cosmeceutically acceptable carrier and an effective amount of vascular endothelial growth factor (VEGF), a VEGF receptor agonist, a prodrug form of the foregoing or a salt form thereof in a non-covalent association complex with a positively-charged backbone having a plurality of attached efficiency groups, the amount being effective for inducing or stimulating new hair growth, increasing hair growth or preventing hair regression in a mammalian subject.

20. The composition of claim 19, wherein the positively-charged backbone comprises a polymer having attached positively charged branching groups.

21. The composition of claim 20, wherein the positively-charged backbone is polylysine.

22. The composition of claim 19, wherein the efficiency group is selected from the group consisting of (gly)ₙ₁(arg)ₙ₂, wherein the subscript n1 is an integer of from about 2 to about 5, and the subscript n2 is an odd integer of from about 7 to about 17, and TAT domains or fragments thereof.

23. The composition of claim 19, wherein the positively charged backbone having a plurality of efficiency groups is a 150,000 to 300,000 MW polylysine backbone having a plurality of attached gly₃arg₇ groups wherein the degree of lysine saturation is from about 5% to about 30%.

24. The composition of claim 19, wherein the carrier further comprises at least one member selected from the group consisting of antimicrobials, moisturizers and hydration agents, penetration agents, preservatives, viscosity-controlling agents and water and optionally including anesthetics, anti-itch actives, botanical extracts, color agents, conditioning agents, darkening or lightening agents, fragrance, glitter, hair pigment additives, humectants, mica, minerals, oils, polyphenols, silicones or derivatives thereof, sunblocks, surfactants, vitamins, waxes, and phytomedicinals.

25. The composition of claim 24, further comprising at least two members selected from the group consisting of antimicrobials, moisturizers and hydration agents, penetration agents, preservatives, viscosity-controlling agents and water.

26. The composition of claim 24, further comprising at least three members selected from the group consisting of antimicrobials, moisturizers and hydration agents, penetration agents, preservatives, viscosity-controlling agents and water.

27. The composition of claim 24, further comprising at least four members selected from the group consisting of antimicrobials, moisturizers and hydration agents, penetration agents, preservatives, viscosity-controlling agents and water.

28. The composition of claim 24, further comprising at least one member selected from the group consisting of antimicrobials, moisturizers and penetration agents and at least one member selected from the group consisting of preservatives, viscosity-controlling agents and water.

29. The composition of claim 24, further comprising at least one member selected from each of the following: antimicrobials, moisturizers, preservatives and water.

30. The composition of claim 24, further comprising at least one moisturizer and at least one preservative.

31. The composition of claim 24, optionally comprising at least one component selected from the group consisting of anesthetics, anti-itch actives, botanical extracts, humectants, silicones or derivatives thereof and phytomedicinals; at least one component selected from the group consisting of conditioning agents, darkening or lightening agents, hair pigment additives, minerals, polyphenols and sunblocks; and at least one component selected from the group consisting of fragrance, glitter, mica, surfactants, vitamins, and waxes.

32. The composition of claim 24, optionally comprising: at least one component selected from the group consisting of anesthetics, anti-itch actives and botanical extracts; at least one component selected from the group consisting of conditioning agents, darkening or lightening agents and hair pigment additives; and at least one component selected from the group consisting of fragrance, surfactants and vitamins.

33. The composition of claim 24, optionally comprising: at least one of each of the following: botanical extracts, conditioning agents and vitamins.

34. The composition of claim 24, wherein the composition is in a form selected from the group consisting of creams and ointments, electroporation formulations, foams, gels, liquids, including solutions, suspensions or emulsions, lotions, microspheres and other degradable microbeads, muds, oils and pastes, ointments, patches, powders, roller sticks, salves, soaps and surfactants, and sprays.

35. The composition of claim 34, wherein the composition is suitable for dispensing from a container selected from the group consisting of bottles, brushes, cans, combs, controlled-release matrices, fabrics, pumps, sprayers, especially aerosol spray dispensers, self-pressurized spray dispensers and non-aerosol spray dispensers, tubes, vials, and wands.

36. The composition of claim 19, wherein the mammalian subject is selected from the group consisting of humans, monkeys, cats, cows, dogs, gerbils, goats, guinea pigs, hamsters, horses, mice, prairie dogs, rabbits, rats, sheep and squirrels.

37. The composition of claim 36, wherein the mammalian subject is a human.

38. The composition of claim 19, wherein the mammalian subject has a condition selected from the group consisting of alopecia, accidental injury, damage to hair follicles, surgical trauma, burn wound, radiation or chemotherapy treatment site, incisional wound, donor site wound from skin transplant, ulcer or desire to modify physical appearance.

39. The composition of claim 19, wherein the composition is in sterile form.

40. The composition of claim 19, wherein the concentration of VEGF is 1.0×10⁻²⁵ M to about 10.0 M.

41. The use of vascular endothelial growth factor (VEGF), a VEGF receptor agonist, a prodrug form of the foregoing or a salt form thereof in non-covalent association with a complex of a positively-charged backbone having a plurality of attached efficiency groups in the preparation of a medicament for topical application to stimulate new hair growth, increase hair growth or prevent hair regression in a mammalian subject

42. A kit for inducing or stimulating new hair growth, increasing hair growth or preventing hair regression in a mammalian subject, the kit comprising a composition and a container, the composition comprising a pharmaceutically or cosmeceutically acceptable carrier and an effective amount of vascular endothelial growth factor (VEGF), a VEGF receptor agonist, a prodrug form of the foregoing or a salt form thereof in non-covalent association with a complex of a positively-charged backbone having a plurality of attached efficiency groups.

43. The kit of claim 42, wherein the composition further comprises at least one member selected from the group consisting of antimicrobials, moisturizers and hydration agents, penetration agents, preservatives, viscosity-controlling agents and water and optionally include anesthetics, anti-itch actives, botanical extracts, color agents, conditioning agents, darkening or lightening agents, fragrance, glitter, hair pigment additives, humectants, mica, minerals, oils, polyphenols, silicones or derivatives thereof, sunblocks, surfactants, vitamins, waxes, and phytomedicinals.

44. The kit of claim 42, wherein the container is selected from the group consisting of bottles, brushes, cans, combs, controlled-release matrices, fabrics, pumps, sprayers, especially aerosol spray dispensers, self-pressurized spray dispensers and non-aerosol spray dispensers, tubes, vials, and wands.

45. The kit of claim 42, wherein the controlled-release matrix delivers the composition over a twenty-four hour period.

46. The kit of claim 45, wherein the controlled-release matrix delivers the composition over an eight hour period.

47. The kit of claim 45, wherein the composition is delivered in a matrix that is not readily washed off or removed with water.
